(19) United States
(12) Patent Application Publication
(10) Pub. No.: US 2026/0193949 A1
(43) Pub. Date: Jul. 9, 2026

(54) SYSTEM AND METHOD FOR PREDICTING GEL STRENGTH OF DRILLING FLUID WHILE DRILLING USING MACHINE LEARNING

(71) Applicant: KING FAHD UNIVERSITY OF PETROLEUM AND MINERALS, Dhahran (SA)

(72) Inventors: Salaheldin Mahmoud Ahmed ELKATATNY, Dhahran (SA); Ahmed Hamdy GOWIDA, Dhahran (SA)

(73) Assignee: KING FAHD UNIVERSITY OF PETROLEUM AND MINERALS, Dhahran (SA)

(21) Appl. No.: 19/008,744

(22) Filed: Jan. 3, 2025

(51) Int. Cl. E21B 21/06; E21B 7/04; E21B 41/00; G01N 9/04; G01N 11/04; G01N 33/28

(52) U.S. Cl. CPC E21B 21/062; E21B 7/046; E21B 41/0035; G01N 9/04; G01N 11/04; G01N 33/2823; E21B 2200/22

(57) ABSTRACT

The present disclosure directs to a computer-implemented method for monitoring and treatment of a drilling fluid during a drilling operation by utilizing machine learning techniques. The method determines a mud density of the drilling fluid at a first predetermined time interval by measuring a mud weight (MW) of the drilling fluid. The method further performs a Marsh funnel (MF) measurement to assess a drilling fluid viscosity of the drilling fluid at the first predetermined time interval. The method further predicts a gel strength of the drilling fluid for a second predetermined time interval in real-time using an artificial neural network based on the measured mud density and the drilling fluid viscosity. The method further adjusts density of the drilling fluid to optimize well cuttings suspension and removal by adding an additive in response to the predicted gel strength.

### STATEMENT REGARDING PRIOR DISCLOSURE BY THE INVENTORS

Aspects of this technology are described in an article “Novel Machine Learning Approach for Predicting the Gel Strength of the Drilling Fluid While Drilling,” This article was presented at the Offshore Technology Conference, Houston, Texas, USA, April 2024, and is herein incorporated by reference in its entirety.

### STATEMENT OF ACKNOWLEDGEMENT

The support provided by the Deanship of Graduate Studies at King Fahd University of Petroleum & Minerals (KFUPM), Dhahran, Saudi Arabia, is gratefully acknowledged.

### BACKGROUND

### Technical Field

The present disclosure is directed to drilling fluid management, and more particularly to systems and methods for predicting gel strength of drilling fluid while drilling by using machine learning techniques.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

In oil and gas drilling operations, drilling fluids are essential for various critical functions that ensure a success of drilling activities. The drilling fluids manage formation pressure, maintain hole cleanliness, and transport drilled cuttings to a surface. Additionally, the drilling fluids serve as lubricants and coolants for a drill string and a drill bit, and form filter cakes to stabilize a wellbore and prevent mud filtration that may otherwise damage a formation. The drilling fluids are broadly classified based on a base fluid type, primarily as oil-based muds or water-based muds. Such classification is crucial in petroleum industry, as each type offers specific properties that meet unique drilling conditions. Mud programs are designed carefully, considering factors like formation sensitivity, fluid activity, section-specific requirements, and safety, to optimize a fluid performance for particular drilling scenarios. Technical formulation of these fluids aims to achieve efficient filtration and rheological characteristics for optimal performance throughout the drilling operations.

Within an oil-based drilling fluid category, synthetic oil-based mud is frequently used for drilling reservoir sections, known for its superior drilling efficiency and reduced risk of formation damage. The synthetic oil-based mud provides significant benefits, such as minimal formation damage and consistent rheological properties during a drilling process. However, a use of the synthetic oil-based mud comes with challenges, including high costs and handling complexities, which needs to be managed effectively in order to maintain operational efficiency.

Monitoring fluid rheology at a drilling site is essential for controlling the fluid performance and ensuring that it meets required operational standards. Fluid rheology measurements provide critical data on properties such as plastic viscosity, yield point, and gel strength, which are continuously monitored by drilling mud crews. Regular rheology measurements are crucial, as they impact mud functions, drilling efficiency, hydraulic behavior, circulation, and pressure management.

Traditional methods for assessing fluid properties include measurement of marsh funnel (MF) viscosity, which records a time required for a certain volume of the fluid to flow through an orifice, and Fann 35 viscometer, which accurately measures mud rheology, including the gel strength, the plastic viscosity, and the yield point, at different rotational speeds such as, 3, 6, 100, 200, 300, and 600 revolutions per minute (rpm). The gel strength is a critical parameter in the drilling fluids, as it prevents sagging, settling, and fluid invasion into formations during static conditions, supporting wellbore stability. Effective control of the gel strength is essential for maintaining wellbore integrity, minimizing risks of wellbore instability, stuck pipes, and fluid loss into porous formations.

Further, gel strength measurements are commonly conducted using a viscometer. A 3-rpm reading is taken after stirring the fluid at 600 rpm to break down any gel structure, followed by a static period of 10 seconds, 10 minutes, and 30 minutes for subsequent readings. Particularly, 30-minute gel strength reading is important, as it indicates whether mud forms a strong gel after prolonged static conditions, such as during tripping out a bottom hole assembly (BHA). Elevated gel strength after the static period may lead to high pump pressure requirements to restore circulation, indicating a need for corrective actions, such as chemical treatments or dilution with fresh base fluid.

The frequency of measuring various fluid properties is tailored to their respective impacts on well control and drilling operations. For instance, mud density and Marsh funnel viscosity properties undergo measurement approximately every hour, typically ranging from three to four times within that timeframe. These frequent evaluations allow for timely adjustments and monitoring of rheological measurements, offering valuable insights into rock sensitivity and the performance of mud activity and stability subsequent to exposure to drilled formations. However, a complete mud test (including all mud rheological properties) is performed twice a day since it consumes considerable time. Without real-time knowledge of, or control over the mud, a control system cannot reliably maintain bottomhole pressure, detect kicks, nor ensure a smooth flow of cuttings. It has been determined that mud rheology, including the yield point, the plastic viscosity, and apparent viscosity, shows strong correlations with fundamental mud properties like mud weight (MW) and the marsh funnel (MF) viscosity.

Conventionally, predictive models have been developed for various rheological properties of the drilling fluids. These models have been tailored to calculate parameters such as, the yield point, the plastic viscosity, and the apparent viscosity using data from drilling fluid compositions. Various mud types, such as the oil-based mud, the water-based mud, invert emulsion mud, water-based Potassium Chloride (KCl) mud, and Calcium Chloride (CaCl₂)) drilling fluid have been modeled to align the fluid properties with drilling requirements. However, these models are limited to specific rheological properties and do not focus on predicting the gel strength, which is a vital aspect of the fluid performance.

US20140291023A1 describes a system that utilizes sensors to measure flow and density to inform adjustments during drilling. Although this system provides immediate feedback on fluid behavior based on real-time measurements, such system lacks predictive capabilities, especially for key properties, such as the gel strength, which are crucial for maintaining the wellbore stability.

Although existing systems provide incremental improvements in monitoring and adjusting the parameters of the drilling fluid, they lack a comprehensive real-time, solution focused on predicting gel strength adjustment based on machine learning. Additionally, a reliance on fixed rule-based models or manual adjustments in prior solutions reduces an ability to quickly adapt to changing wellbore conditions. These gaps in existing technologies highlight a need for a more advanced, and an adaptable system that leverages machine learning to predict and manage drilling fluid properties dynamically.

Accordingly, it is one object of the present disclosure to provide methods and systems for real-time monitoring, prediction, and adjustment of the drilling fluid properties, particularly the gel strength, during the drilling operations. The present disclosure achieves this by utilizing machine learning models such as, an artificial neural network (ANN) model that predict the gel strength at regular time intervals shorter than gel strength measurement time intervals. Prediction of the gel strength is based on measurements like, the mud weight (MW) and the marsh funnel (MF) viscosity. This predictive approach enables proactive adjustments to the fluid composition, enhancing the wellbore stability, optimizing cuttings removal, and improving the drilling efficiency.

### SUMMARY

In an exemplary embodiment, a computer-implemented method for monitoring and treatment of drilling fluid during a drilling operation is disclosed. The method includes measuring the mud weight (MW) of the drilling fluid to determine the mud density of the drilling fluid at a first predetermined time interval. The method further includes performing a marsh funnel (MF) measurement to determine a drilling fluid viscosity of the drilling fluid at the first predetermined time interval. The method further includes predicting the gel strength of the drilling fluid for a second predetermined time interval in real-time by an artificial neural network based on the mud density and the drilling fluid viscosity. The method further includes adjusting the density of the drilling fluid to control suspension and removal of well cuttings by adding an additive based on the predicted gel strength.

In another exemplary embodiment, a computer-implemented system for monitoring and treatment of a drilling fluid during a drilling operation is disclosed. The system includes a drilling rig configured to perform the drilling operation with the drilling fluid. The system further includes a mud balance to measure mud weight (MW) of the drilling fluid at a first predetermined time interval. The system further includes a marsh funnel (MF) to determine a drilling fluid viscosity of the drilling fluid at the first predetermined time interval. The system further includes processing circuitry configured with an artificial neural network to predict a gel strength of the drilling fluid fort a second predetermined time interval in real-time based on the mud weight (MW) and the drilling fluid viscosity. The system is configured to adjust density of the drilling fluid to control suspension and removal of well cuttings with a dispenser for adding an additive in accordance with the predicted gel strength.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates a block diagram of a system for monitoring and treatment of drilling fluid during a drilling operation, according to certain embodiments.

FIG. 1B illustrates a schematic representation of a drilling rig, according to certain embodiments.

FIG. 1C illustrates a schematic representation of a mud balance, according to certain embodiments.

FIG. 1D illustrates a schematic representation of a marsh funnel (MF), according to certain embodiments.

FIG. 2 illustrates components of a processing circuitry of a server, according to certain embodiments.

FIG. 3 illustrates an exemplary schematic representation of artificial neural network (ANN) model for predicting gel strength of the drilling fluid, according to certain embodiments.

FIG. 4 illustrates a graph showing correlation coefficients (R) between model input parameters and model output, according to certain embodiments.

FIGS. 5A-5B illustrates a graph representing a comparison between predicted gel strength measurements and actual gel strength measurements for training data, according to certain embodiments.

FIGS. 6A-6B illustrates a graph representing a comparison between predicted gel strength measurements and actual gel strength measurements for testing data, according to certain embodiments.

FIG. 7 illustrates a flowchart of a method for developing a computing model, according to certain embodiments.

FIG. 8 illustrates a flowchart of a method for predicting the gel strength of and treatment of the drilling fluid, according to certain embodiments.

FIG. 9 is an illustration of a non-limiting example of details of a computing hardware used in a computing system, according to certain embodiments.

FIG. 10 is an exemplary schematic diagram of a data processing system used within the computing system, according to certain embodiments.

FIG. 11 is an exemplary schematic diagram of a processor used with the computing system, according to certain embodiments.

FIG. 12 is an illustration of a non-limiting example of distributed components which may share processing with a controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a system and a method for improving an estimation of gel strength of drilling fluid to enhance drilling operations and avoid settings of cuttings in a wellbore. Conventional measurements of the gel strength typically use rotational viscometers, which are limited by corresponding time intensive nature, equipment dependency, and lack of real-time monitoring. For example, a complete mud test (including all mud rheological properties) is performed twice a day since it consumes considerable time.

The present disclosure relates to a system and a method that applies machine learning (ML) models using accessible drilling parameters, such as mud weight (MW) and marsh funnel (MF) viscosity, as an input to predict the gel strength at time intervals that are shorter than gel strength would be measured. The machine learning (ML) models such as, neural networks may be constructed using a dataset of surface drilling parameters and laboratory gel strength values to predict the gel strength at a regular time interval with a high precision, promoting enhanced drilling performance, operational safety, and automation potential within drilling workflows.

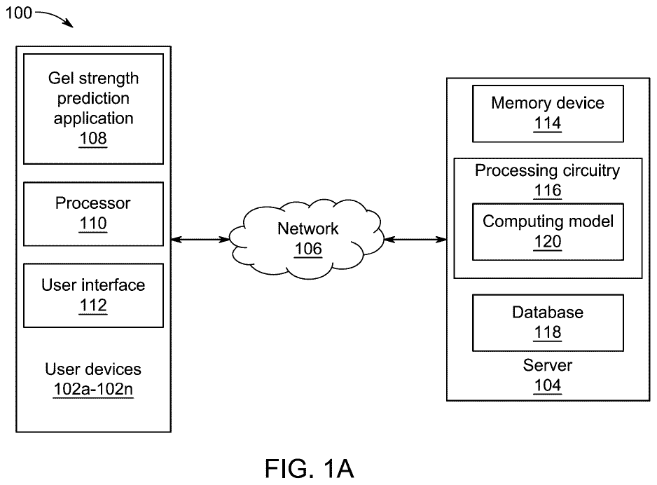

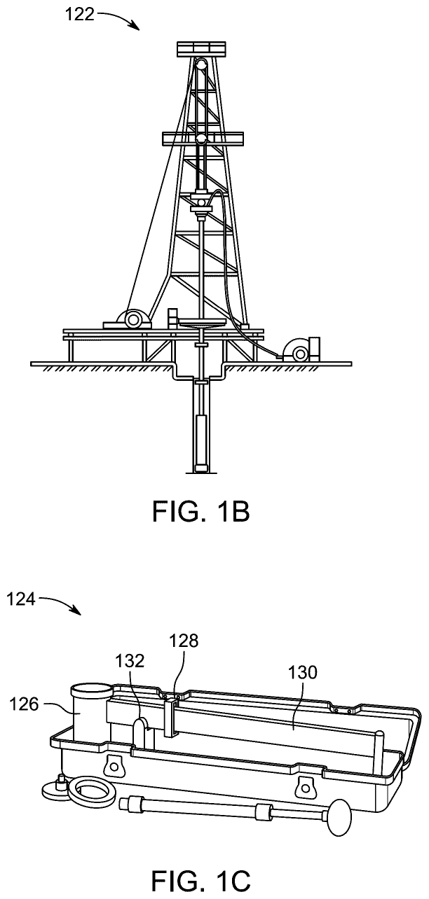

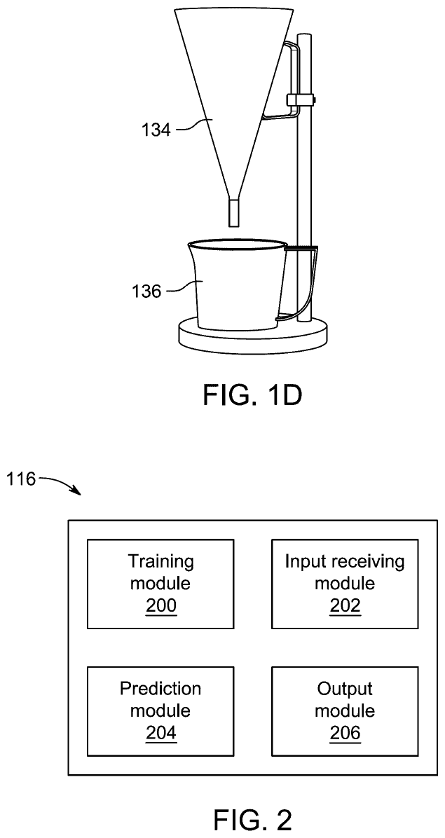

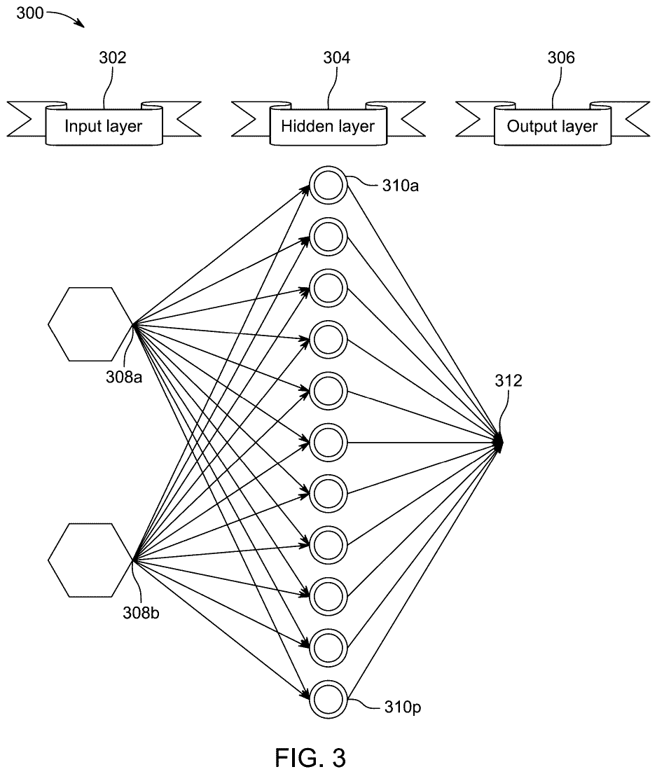

FIG. 1A illustrates a block diagram of a system 100 for monitoring and treatment of drilling fluid during a drilling operation, according to certain embodiments. As used herein, the “drilling fluid” may be referred to as drilling mud that is a mixture of liquids, solids, or both and circulated around a drill bit while performing the drilling operation. In an embodiment, the drilling fluid may be, but not limited to, water-based mud, weighted mud, foam drilling mud, and so forth. In a preferred embodiment, the drilling fluid may be synthetic oil-based mud. Embodiments of the present invention are intended to include or otherwise cover any type of the drilling fluid including known, related art, and/or later developed technologies.

The drilling operation may be, but not limited to, drilling oil wells, drilling gas wells, and the like. According to embodiments of the present disclosure, the system 100 may be configured to estimate gel strength of the drilling fluid, that may be crucial for keeping drilling cuttings suspended in a wellbore and avoiding potential blockages. The system 100 may also be configured to offer real-time monitoring and precise predictions of the gel strength to enhance drilling efficiency, safety, and automation initiatives. As used herein, the term “gel strength of the drilling fluid” refers to a rheological property that measures a shear stress of the drilling mud at a low shear rate after the drilling mud has been at rest for a specified period of time.

In an embodiment, the system 100 may include one or more user devices 102a-102n (hereinafter collectively referred to as the user devices 102 and individually referred to as the user device 102) and a server 104. In such embodiment, the user device 102 and the server 104 may be connected to each other through a network 106.

According to an embodiment, the network 106 may be a data network such as, but not limited to, the Internet, a local area network (LAN), a wide area network (WAN), a metropolitan area network (MAN), and so forth. Embodiments of the present invention are intended to include or otherwise cover any type of the data network, including known, related art, and/or later developed technologies. In some embodiments, the network 106 may be a wireless network, such as, but not limited to, a cellular network and may employ various technologies including an enhanced data rates for global evolution (EDGE), a general packet radio service (GPRS), and so forth. Embodiments of the present invention are intended to include or otherwise cover any type of the wireless network, including known, related art, and/or later developed technologies.

Further, the user device 102 may be a device used by an operator for uploading measured values of drilling fluid parameters. In an embodiment, the drilling fluid parameters may be, but not limited to, mud weight (MW), drilling fluid viscosity, and so forth. As used herein, the term “mud weight (MW)” refers to a density of the drilling fluid and is normally measured in pounds per gallon (lb/gal) (ppg) or pound cubic feet (pcf). Also, as used herein, the term “drilling fluid viscosity” refers to a measure of how resistant the drilling fluid is to flow. The drilling fluid viscosity is defined as a ratio of shear stress to shear rate and is measured in poises or centipoises (cP). Embodiments are intended to include or otherwise cover any drilling fluid parameters that may be utilized for predicting the gel strength of the drilling fluid. In an embodiment, the drilling fluid parameters may be measured by using one or more measuring tools such as, a mud balance 124 (as shown and explained in detail in FIG. 1C) and a marsh funnel (MF) 134 (as shown and explained in detail in FIG. 1D). In another embodiment, the drilling fluid parameters may be measured by using one or more sensors (not shown) placed in a drilling fluid system. In an embodiment, the drilling fluid parameters may be measured at a first predetermined time interval.

The first predetermined time interval for measuring mud weight (MW) and performing Marsh Funnel (MF) viscosity measurements typically ranges from 10 to 15 minutes, depending on the operational requirements. This interval begins at the start of a monitoring cycle (e.g., 0 minutes) and ends with the next scheduled measurement within the defined range (e.g., 10-15 minutes later). The specific interval may be adjusted based on drilling conditions and the frequency required for effective fluid management.

The user device 102 may be for example, but not limited to, a mobile device, a smart phone, a tablet, a portable computer, a laptop, a desktop, a smart device, and so forth. Embodiments are intended to include or otherwise cover any type of the user device 102, including known, related art, and/or later developed technologies. Further, the user device 102, as may be readily appreciated by a person skilled in the art, is merely intended to illustrate and not to limit what may encompass the user device 102, such as, but not limited to, an instant messaging sending device, a short message service (SMS) transmitting device, and/or other messaging devices that may include, but not limited to, a text, graphics, symbols and/or other identifiable communications. In an embodiment, the user device 102 may be a multipurpose device, such that an operation in accordance with the present system 100 is merely one of many (e.g., two or more) features that may be provided by the user device 102.

According to an embodiment, the user device 102 may include software applications such as, but not limited to, a navigation application, a camera application, a media player application, a social networking application, and so forth. In a preferred embodiment, the user device 102 may include a gel strength prediction application 108 that may be a computer readable program installed on the user device 102 for executing functions associated with the system 100 on the user device 102. In an embodiment, the operator may login into the system 100 through the gel strength prediction application 108 by providing login details such as, but not limited to, a user identifier, a passcode, and so forth. Embodiments of the present invention are intended to include or otherwise cover any type of the login details that may be associated with the operator. Upon login into the system 100, the operator may input the measured values of the drilling fluid parameters onto the system 100 using the gel strength prediction application 108. In another embodiment, the user may login into the system 100 through a web browser by providing the login details. In such embodiment, the user may input the measured values of the drilling fluid parameters onto the system 100 using the web browser upon login into the system 100.

The user device 102 may further comprise a processor 110 and a user interface 112. The processor 110 may be configured to receive and/or transmit data associated with the system 100 over the network 106. Further, the processor 110 may be configured to process the data associated with the system 100, in an embodiment of the present invention. The processor 110 may be, but not limited to, a programmable logic control unit (PLC), a microcontroller, a microprocessor, a computing device, a development board, and so forth. Embodiments of the present invention are intended to include or otherwise cover any type of the processor 110 including known, related art, and/or later developed technologies.

The user interface 112 may be configured to enable the operator to interact with the gel strength prediction application 108 installed within the user device 102. The user interface 112 may be further configured to display output data associated with the system 100. In an embodiment, the output data may be predicted gel strength measurements of the drilling fluid. The user interface 112 may be, but not limited to, a digital display, a touch screen display, a graphical user interface, and so forth. Embodiments of the present invention are intended to include or otherwise cover any type of the user interface 112 including known, related art, and/or later developed technologies.

In an embodiment, the server 104 may include a memory device 114, a processing circuitry 116, and a database 118. The memory device 114 may be a non-transitory data storage medium that may be configured to store computer executable instructions for controlling operations of the system 100. The memory device 114 may be, but not limited to, a random-access memory (RAM) device, a read only memory (ROM) device, a flash memory, and so forth. Embodiments of the present invention are intended to include or otherwise cover any type of the memory device 114 including known, related art, and/or later developed technologies.

Further, the processing circuitry 116 may be connected to the memory device 114 to execute the computer executable instructions to perform the operations associated with the system 100. The processing circuitry 116 may be, but not limited to, the programmable logic control unit (PLC), the microcontroller, the microprocessor, the computing device, the development board, and so forth. Embodiments of the present invention are intended to include or otherwise cover any type of the processing circuitry 116 including known, related art, and/or later developed technologies.

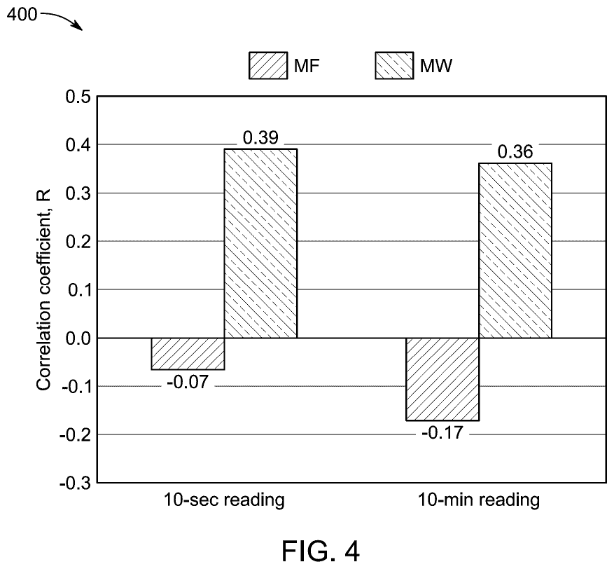

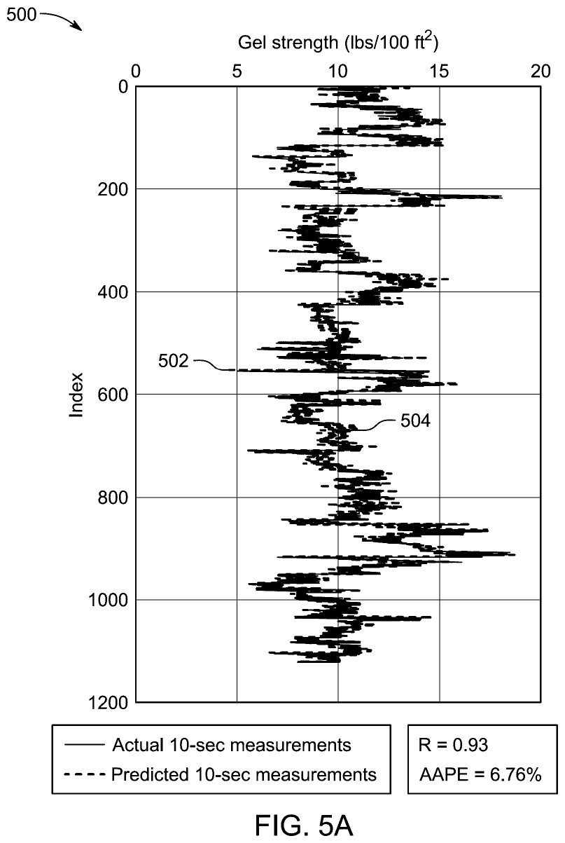

In an embodiment, the processing circuitry 116 may be configured with a computing model 120 that may be trained for predicting the gel strength of the drilling fluid in real-time. In a preferred embodiment, the computing model 120 may be a machine learning (ML) model such as an artificial neural network (ANN) model 300 (as shown in FIG. 3). In an embodiment, the computing model 120 may predict the gel strength of the drilling fluid based on the measured values of the drilling fluid parameters received from the user device 102. In an embodiment, the processing circuitry 116 may be explained in detail in conjunction with FIG. 2.

In an embodiment, the database 118 may be configured to store one or more datasets (hereinafter collectively referred to as the datasets and individually referred to as the dataset). The dataset may include historical measured values of the drilling fluid parameters and laboratory gel strength measurements of the drilling fluid (hereinafter referred to as the actual gel strength measurements). The historical measured values of the drilling fluid parameters and the actual gel strength measurements may be collected from mud reports and stored in the database 118 as the dataset. As used herein, the term “mud reports” refers to documents used in a drilling industry to record and analyse properties of the drilling fluid during the drilling operation. Also, as used herein, the term “laboratory gel strength measurements” refers to empirical data points obtained from controlled experiments that measure the gel strength of the drilling fluid. In an embodiment, a statistical analysis may be conducted on the historical measured values of the drilling fluid parameters and the actual gel strength measurements, as shown in Table 1 below.

TABLE 1
Data Statistical Analysis

| | |
|---|---|
| Inputs | Outputs |

| | Mud | Marsh | Gel Strength | Gel Strength |
|---|---|---|---|---|
| | Weight | Funnel | (10 Sec | (10-Min |
| Statistical | (MW) | (MF) | Reading) | Reading) |
| Parameter | (lbs./ft³) | (sec⁻¹) | (Ibs./100 ft²) | (Ibs./100 ft²) |
| Minimum | 63.0 | 26.0 | 5.0 | 9.0 |
| Maximum | 140.0 | 120.0 | 18.0 | 38.0 |
| Range | 77.0 | 94.0 | 23.0 | 38.0 |
| Mean | 98.1 | 66.0 | 10.5 | 17.6 |
| Standard | 13.9 | 10.8 | 2.6 | 5.3 |
| Deviation | | | | |
| Skewness | 0.4 | 0.5 | 0.0 | 0.7 |

Table 1 illustrates key statistical parameters that provide insights into a distribution and a variability of the dataset and may be used to train and test the computing model 120. The statistical parameters such as, minimum and maximum values indicate a range of each variable, showing smallest and largest recorded measurements across the dataset. For example, the mud weight (MW) ranges from 63.0 to 140.0 lbs./ft³, inputs of the marsh funnel (MF) 134 ranges from 26.0 to 120.0 lbs./ft³, 10-second gel strength measurements range from 0.0 to 23.0 lbs./100 ft², and 10-minute gel strength measurements range from 0.0 to 38.0 lbs./100 ft².

Further, the statistical parameters such as range represents a difference between the maximum and minimum values of each parameter. The range shows a variability of the values within the dataset for the corresponding parameter that may provide insights onto how much a particular parameter varies. For example, if the mud weight (MW) is having a minimum value of 63.0 and a maximum value of 140.0, then the range=maximum−minimum=140.0−63.0=77.0, which helps assess the variability in the dataset.

The statistical parameter such as, mean provides an average of values of each parameter, giving a central reference point. For instance, mean gel strength at 10-second interval is 10.5 lbs./100 ft², while at 10-minute interval, the mean increases to 17.6 lbs./100 ft², indicating an expected increase in the gel strength over time.

Further, the statistical parameter such as, a standard deviation indicates how much the values deviate from the mean. The standard deviation of the gel strength such as, 2.6 for the 10-second interval and 5.3 for 10-minute interval suggests moderate variability.

Also, skewness values reveal whether the distribution of the parameter is symmetrical. For example, a slight positive skew in the input of the marsh funnel (MF) 134 i.e. 0.5 suggests a tail of higher values which a model may need to account for when predicting the gel strength.

In another embodiment, the server 104 may include multiple databases (not shown) to store the dataset. According to embodiments of the present invention, the database 118 may be for example, but not limited to, a centralized database, a distributed database, a personal database, an end-user database, a commercial database, a structured query language (SQL) database, a non-SQL database, an operational database, a relational database, a cloud database, an object-oriented database, a graph database, and so forth. Embodiments of the present invention are intended to include or otherwise cover any type of the database 118 including known, related art, and/or later developed technologies that may be capable of data storage and retrieval.

FIG. 1B illustrates a schematic representation of a drilling rig 122, according to certain embodiments. The drilling rig 122 may be configured to perform the drilling operation with the drilling fluid. In an embodiment, the drilling rig 122 may be configured to perform the drilling operation in a vertical well. In another embodiment, the drilling rig 122 may be configured to perform the drilling operation in a horizontal well. In yet another embodiment, the drilling rig 122 may be configured to perform the drilling operation in an inclined well. In another embodiment, the drilling rig 122 may be configured to perform the drilling operation in a multilateral well, drilling operation in combinations of vertical, horizontal, and inclined.

FIG. 1C illustrates a schematic representation of the mud balance 124, according to certain embodiments. As used herein, the term “mud balance 124” refers to a rugged and an easily calibrated instrument that is suitable for measuring the drilling fluid parameters such as, the mud weight (MW) (density). In an embodiment, the mud balance 124 may be an essential tool in the drilling operation, enabling accurate assessments of the mud weight (MW) of the drilling fluid to optimize performance and safety. In an embodiment, a method of utilizing the mud balance 124 to determine the mud weight (MW) may include an initial step of filling a cup 126 of the mud balance 124 with the drilling mud to be tested by the operator. The initial step ensures that a sufficient quantity of the drilling fluid is available for accurate measurement. The method further includes a step of placing a lid (not shown) securely on the cup 126 to prevent spillage and wiping off excess drilling mud to ensure that only the drilling mud within the cup 126 is measured. The method further includes a step of moving a rider 128 of an arm 130 of the mud balance 124 along a length until a balance is achieved. This adjustment is critical for achieving an accurate reading. The method further includes a step of reading the density of the drilling mud by the operator by observing a scale at a side of the rider 128 that is closest to a knife edge 132. The reading provides the operator with the mud weight (MW), that may be crucial for maintaining an appropriate pressure balance in the wellbore.

FIG. 1D illustrates a schematic representation of the marsh funnel (MF) 134, according to certain embodiments. As used herein, the term “marsh funnel (MF) 134” refers to a tool adapted to measure the drilling fluid viscosity in a field. The marsh funnel (MF) 134 may provide a practical and an efficient method for determining flow characteristics of the drilling fluid, which is critical for ensuring the performance of the drilling fluid in wellbore operations. In an embodiment, a method of utilizing the marsh funnel (MF) 134 to measure the drilling fluid viscosity may include an initial step of filling the marsh funnel (MF) 134 with the drilling fluid by the operator, ensuring that any particles or debris that may obstruct a flow of the marsh funnel (MF) 134 are filtered out. The method further includes a step of holding the marsh funnel (MF) 134 in a vertical orientation, keeping an end of a tube of the marsh funnel (MF) 134 sealed with a finger. The marsh funnel (MF) 134 may be positioned above a measuring cup 136 to collect the drilling fluid as the drilling fluid flows through the tube. The method further includes a step of releasing the finger from the end of the tube, once the marsh funnel (MF) 134 is in place, allowing the drilling fluid to flow freely. Simultaneously, a timing device, such as a stopwatch, is started to record a time taken for the drilling fluid to flow through the marsh funnel (MF) 134 into the measuring cup 136.

FIG. 2 illustrates components of the processing circuitry 116 of the server 104, according to certain embodiments. The components may be a training module 200, an input receiving module 202, a prediction module 204 and an output module 206.

According to an embodiment, the training module 200 may be configured to train the computing model 120 (as shown in the FIG. 1A) by using the dataset stored in the database 118 (as shown in the FIG. 1A). In an embodiment, the computing model 120 may be the machine learning (ML) model. The machine learning (ML) model may be, but not limited to, a linear regression model, a logistic regression model, a support vector regression model, a random forest model, a boosted tree model, a multi-layer perceptron model, a conventional neural network model, a fuzzy logic model, and so forth. In a preferred embodiment, the machine learning (ML) model may be the artificial neural network (ANN) model 300 (as shown in the FIG. 3). Embodiments of the present invention are intended to include or otherwise cover any type of machine learning (ML) model including known, related art, and/or later developed technologies. A dataset can include Marsh Funnel (MF) viscosity and mud weight (MW) as input features, paired with corresponding gel strength measurements for both 10-second and 10-minute intervals as target outputs. These features are collected simultaneously, ensuring consistent intervals between input and target data. Gel strength measurements at the 10-second and 10-minute intervals are obtained using a rotational viscometer (e.g., Fann 35). After stirring the fluid to break its gel structure, readings are taken after static periods of 10 seconds and 10 minutes. This method is consistent and ensures the reliability of the target data used for training.

In an embodiment, the training module 200 may be configured to retrieve the dataset containing the historical measured values of the drilling fluid parameters and the actual gel strength measurements from the database 118. In an exemplary embodiment, the historical measured values of the drilling fluid parameters may be used as model inputs due to accessibility and cost-effectiveness of these drilling fluid parameters and the actual gel strength measurements may be considered as a target output. In an embodiment, the training module 200 may be configured to establish a connection with the database 118 for retrieving the dataset. In such embodiment, the training module 200 may be configured to establish the connection with the database 118 upon receiving a command to initiate a model training from the user device 102 (as shown in the FIG. 1A). In an embodiment, the connection is managed by an interface that enables an efficient transfer. Upon establishing the connection with the database 118, the training module 200 may be configured to generate and transmit a query, specifying the historical measured values of the drilling fluid parameters and the actual gel strength measurements for the model training to the database 118.

Upon receiving the dataset containing the historical measured values of the drilling fluid parameters and the actual gel strength measurements from the database 118, the training module 200 may be configured to process the dataset for removing errors, duplicates, inconsistencies, and so forth by using data cleaning techniques. In an embodiment, the training module 200 may be configured to remove the errors, the duplicates, the inconsistencies, and so forth by handling missing values, outliers, and noisy data in the dataset. The data cleaning techniques may be, but not limited to, statistical techniques, smoothing techniques, filtering techniques, and so forth. Embodiments of the present invention are intended to include or otherwise cover any type of the data cleaning techniques including known, related art, and/or later developed technologies.

Further, in an embodiment, the training module 200 may be configured to normalize the dataset by using normalization techniques. In such embodiment, the normalization techniques may be, but not limited to, a min-max scaling, a z-score standardization, and so forth. Embodiments of the present invention are intended to include or otherwise cover any type of the normalization techniques including known, related art, and/or later developed technologies. In an embodiment, the training module 200 may be configured to normalize the dataset for maintaining comparability, if measurements of the drilling fluid parameters differ in units or scales.

The training module 200 may further be configured to transform the dataset into meaningful representations, enhancing a learning ability of the computing model 120. Upon transforming the dataset, the training module 200 may be configured to obtain relevant features from the dataset by using a feature selection technique. In an exemplary embodiment, the training module 200 may be configured to obtain the relevant features such as, the mud weight (MW) and the drilling fluid viscosity from the dataset. In an alternative embodiment, features may be determined using a feature selection technique which may include, but not limited to, permutation feature importance, stochastic feature selection, Boruta technique, and so forth. In a preferred embodiment, the feature selection technique may be a relative importance study that evaluates an impact of each of the features present in the dataset on predicting target values such as, the gel strength measurements, ensuring that only significant features are retained. Embodiments of the present invention are intended to include or otherwise cover any type of the feature selection technique including known, related art, and/or later developed technologies.

Further, in an embodiment, the training module 200 may be configured to stratify the processed dataset into training data, testing data and validation data by using a stratification technique. As used herein, the term “training data” refers to a portion of the data reserved to fit the computing model 120. In other words, the computing model 120 sees and learns from the data in the training data to directly improve the parameters. Also, as used herein, the term “testing data” refers to a set of data used to evaluate a final performance of a trained model. Further, as used herein, the term “validation data” refers to the set of data used to evaluate and fine-tune the model during training, helping to assess the performance of the model and make adjustments.

The stratification technique may be, but not limited to, a random sampling, a cross validation splitting, and so forth. In a preferred embodiment of the present invention, the stratification technique may be a stratified dataset splitting. As used herein, the term “stratified dataset splitting” refers to a method commonly used with imbalanced datasets, where certain classes or categories have significantly fewer instances than others. In such cases, it is crucial to ensure that the training data, the validation data, and testing data adequately represent a class distribution to avoid bias in a final model. Embodiments of the present invention are intended to include or otherwise cover any type of the stratification technique including known, related art, and/or later developed technologies.

Further, in an embodiment, the training module 200 may be configured to train the computing model 120 using the training data based on default hyperparameters for generating a baseline computing model 120. The baseline computing model 120 may help in establishing a performance benchmark for comparison for subsequent optimization steps. For example, initially the computing model 120 such as, the artificial neural network (ANN) model 300 is trained with one hidden layer 304 (as shown in the FIG. 3) and the default hyperparameters to predict the gel strength of the drilling fluid using the historical measured values of the drilling fluid parameters such as, the mud weight (MW) and the drilling fluid viscosity.

Further, the training module 200 may be configured to determine one or more combinations of hyperparameters for training the computing model 120 on each of the combinations of the hyperparameters. In an exemplary embodiment, the hyperparameters may be, but not limited to, a learning rate, number of hidden layers 304, number of neurons 310a-310p (as shown in the FIG. 3) per hidden layer 304, transfer functions, training algorithms, and so forth. Embodiments of the present invention are intended to include or otherwise cover any type of the hyperparameters.

In an embodiment, the learning rate may be in a range of 0.01 to 0.9 and the number of hidden layers 304 may be in a range of 1 to 4, where each hidden layer 304 may be having the neurons 310a-310p in a range of 5 to 30. Further, the transfer functions may be, but not limited to, a log-sigmoid (log sig) transfer function, a hyperbolic tanget sigmoid (tansig) transfer function, a radial basis (radbas) transfer function, a pure-linear (purelin) transfer function, an elliot symmetric sigmoid (elliotsig) transfer function, a saturating linear (satlin) transfer function, a triangular basis (tribas) transfer function, a hard limit (hardlim) transfer function, and so forth. Embodiments of the present invention are intended to include or otherwise cover any type of the transfer functions including known, related art, and/or later developed technologies.

The training algorithms may be, but not limited to, levenberg-marquardt backpropagation (trainlm), resilient backpropagation (trainrp), gradient descent backpropagation (traingd), gradient descent with adaptive learning rule backpropagation (traingda), bayesian regularization (trainbr), cyclical order incremental update (trainc), and so forth. Embodiments of the present invention are intended to include or otherwise cover any type of the training algorithms including known, related art, and/or later developed technologies.

In an embodiment, the training module 200 may be configured to determine the combinations of the hyperparameters by using an optimization technique to train the computing model 120 on each of the combinations of the hyperparameters. The optimization technique may be, but not limited to, a random search, a bayesian optimization, and so forth. In a preferred embodiment, the optimization technique may be a grid search function. Embodiments of the present invention are intended to include or otherwise cover any type of the optimization technique including known, related art, and/or later developed technologies. In an embodiment, the training module 200 may be configured to assess a performance of the trained computing model 120 on each of the combinations of the hyperparameters to identify optimal combination of the hyperparameters.

For example, for the artificial neural network (ANN) model 300, different combinations of the hyperparameters are tested, including different numbers of hidden layers 304 such as, 1, 2, 3, varying counts per layer such as, 10, 20, 30, the learning rates such as, 0.01, 0.1, 0.2, the transfer functions such as, the tanget sigmoid (tansig) transfer function, the pure-linear (purelin) transfer function, and the log-sigmoid (log sig) transfer function, and the training algorithms such as, the levenberg-marquardt backpropagation (trainlm), the resilient backpropagation (trainrp) and the bayesian regularization (trainbr) are tested. Through this process, if the artificial neural network (ANN) model 300 with a single hidden layer 304 containing 18 neurons 310a-310p, the levenberg-marquardt backpropagation (trainlm) algorithm, 0.1 learning rate, the log-sigmoid (log sig) transfer function between an input layer 302 (as shown in the FIG. 3) of the artificial neural network (ANN) model 300 and the hidden layer 304 of the artificial neural network (ANN) model 300 and the pure-linear (purelin) transfer function between the hidden layer 304 of the artificial neural network (ANN) model 300 and an output layer 306 (as shown in the FIG. 3) of the artificial neural network (ANN) model 300 yields a lowest error, then this optimal combination of the hyperparameters is selected for further tuning.

In an embodiment, the training module 200 may further be configured to train the computing model 120 on the training data with the optimal combination of the hyperparameters. During a training process, the training module 200 may be configured to calculate an error objective function after each training iteration for performing weight adjustments within the computing model 120. As used herein, the term “error objective function” refers to a mathematical formula that calculates a difference between a current prediction of the model and actual values in the dataset. In an embodiment, the error objective function may include, but is not limited to, a mean squared error (MSE), a mean absolute error (MAE), a cross-entropy loss, and so forth. Embodiments of the present invention are intended to include or otherwise cover any type of the error objective function including known, related art, and/or later developed technologies.

For example, in a first iteration, the artificial neural network (ANN) model 300 predicts the gel strength of the drilling fluid for a first batch of the training data. For each prediction, the mean squared error (MSE) is calculated by taking a difference between the actual gel strength measurements and the predicted gel strength measurements, squaring a result of the difference, and averaging across all samples in the first batch of the training data. Suppose the mean squared error (MSE) after the first iteration is 0.15 which is high indicating that the predicted gel strength measurements are far from the actual gel strength measurements. The artificial neural network (ANN) model 300 propagates an error, adjusting weights within the neurons 310a-310p based on the training algorithm to reduce the mean squared error (MSE).

Further, in a second iteration, with the weight adjustments, the artificial neural network (ANN) model 300 predicts new gel strength measurements. The mean squared error (MSE) is recalculated based on the new gel strength measurements, resulting in a lower error of, for example, 0.10. Hence, through iterative recalculation of the mean squared error (MSE) and the weight adjustments, the artificial neural network (ANN) model 300 converges to an optimal state where the error objective function reaches a minimum threshold, signifying that the artificial neural network (ANN) model 300 effectively learned input-output relationships within the training data. The training module 200 may be configured to update the hyperparameters of the artificial neural network (ANN) model 300 based on the calculated error objective function to improve a prediction accuracy.

The artificial neural network model 300 can predict gel strength values for both 10-second and 10-minute intervals corresponding to each pair of measured MW and MF values. The predictions are synchronized with the intervals at which MW and MF are measured. The gel strength predictions are made at the same intervals as the MW and MF measurements. Typically, MW and MF measurements occur every 10-15 minutes during operations. Accordingly, the model predicts gel strength (both 10-second and 10-minute values) at the same 10-15 minute intervals. This ensures that the predictions are timely and actionable for operational adjustments.

In particular, the output of the neural network model is a single gel strength measurement value for the 10-second interval and another for the 10-minute interval, corresponding to each set of input parameters (mud weight and Marsh Funnel viscosity). The model provides these discrete predictions at the intervals when the input measurements are taken.

While the logs may display these predictions as a line graph over time, this visualization represents consecutive independent predictions rather than a continuous sequence. Each data point corresponds to a specific input set and its associated outputs.

In an exemplary embodiment, the training module 200 may be configured to feed the testing data into the trained computing model 120 to predict the target output for a second predetermined time interval. The second predetermined time interval for predicting gel strength corresponds directly to the timing of MW and MF measurements. The gel strength is predicted for both 10-second and 10-minute intervals after static conditions, aligning with standard gel strength measurement practices in drilling operations.

The training module 200 may be configured to apply the trained computing model 120 to the testing data to evaluate the performance of the computing model 120 on an unseen data. The training module 200 may be configured to record performance metrics such as, but not limited to, a correlation coefficient (R), an average absolute percentage error (AAPE), and so forth for evaluating the prediction accuracy of the computing model 120. For instance, the testing data consists of 455 data points of measurements of the mud weight (MW) and the drilling fluid viscosity. The computing model 120 such as the artificial neural network (ANN) model 300 may use the data points to predict the gel strength at the second predetermined time interval. Upon testing, the artificial neural network (ANN) model 300 may show the correlation coefficient (R) exceeding 0.93 and the average absolute percentage error (AAPE) values of 7.08% for the 10-second interval and 7.21% for the 10-minute interval gel strengths, indicating high alignment with the actual gel strength measurements.

Further, in an embodiment, the training module 200 may be configured to analyze a testing performance of the computing model 120 by reviewing values of the performance metrics. If the performance metrics do not meet predefined accuracy thresholds, then the training module 200 may be configured to perform iterative refinements such as, a hyperparameter adjustment, a model architecture tuning, and so forth until the computing model 120 achieves a desired performance. Once the iterative refinements are completed and the desired performance is achieved, then the training module 200 may be configured to develop the computing model 120 with a final model configuration. The final model configuration may include the optimized hyperparameters, feature set, the performance metrics, and so forth.

In a preferred embodiment, the computing model 120 is an artificial neural network (ANN) model 300 with a configuration including the single hidden layer 304 containing the 18 neurons 310a-310p, the levenberg-marquardt backpropagation (trainlm) algorithm with the learning rate of 0.10, utilizing the log-sigmoid (log sig) transfer function between the input layer 302 and the hidden layers 304, and the pure-linear (purelin) transfer function between the hidden layer 304 and the output layer 306. The training module 200 may document the configuration as an optimized model configuration that may be ready for deployment in real-world applications.

Further, the training module 200 may be configured to validate the computing model 120 using the validation data for confirming a predictive performance on new data, ensuring no overfitting has occurred. The training module 200 may be configured to validate the computing model 120 by calculating the performance metrics such as, but not limited to, the correlation coefficient (R) and the average absolute percentage error (AAPE) to ensure that the predictive accuracy of the computing model 120 meets a predefined deployment criterion.

The input receiving module 202 may be configured to receive the values of the drilling fluid parameters measured using the measurement tools from the user device 102 (as shown in the FIG. 1A). In an exemplary embodiment, the drilling fluid parameters may be the mud density of the drilling fluid that may be determined by measuring the mud weight (MW) of the drilling fluid. In such embodiment, the mud weight (MW) of the drilling fluid may be measured by using the mud balancer 124 (as shown in the FIG. 1C) at the first predetermined time interval. In an embodiment, the first predetermined time interval may be every hour.

In particular, the artificial neural network predicts discrete gel strength values directly linked to the input parameters collected at that specific interval. The gel strength predictions are determined for each time interval at which mud weight (MW) and Marsh Funnel (MF) viscosity measurements are taken. If MW and MF measurements are collected at regular intervals, such as every hour, the model provides a single gel strength prediction for the 10-second interval and another for the 10-minute interval corresponding to each measurement event.

Subsequently, gel strength predictions are generated at the same intervals as MW and MF measurements, typically every 10-15 minutes or at other regular intervals, depending on the operational setup. The model provides discrete gel strength values—one for the 10-second interval and another for the 10-minute interval—corresponding to each MW and MF measurement. These outputs are specific to the inputs at the time of measurement.

In another exemplary embodiment, the drilling fluid parameters may be the drilling fluid viscosity that may be determined by performing a marsh funnel (MF) measurement. In such embodiment, the marsh funnel (MF) measurement may be performed by using the marsh funnel (MF) 134 (as shown in the FIG. 1D) at the first predetermined time interval. The input receiving module 202 may be configured to transmit the received measurements of the drilling fluid parameters to the prediction module 204.

The prediction module 204 may be communicatively coupled to the input receiving module 202. The prediction module 204 may be configured to receive the measurements of the drilling fluid parameters from the input receiving module 202. The prediction module 204 may be configured to feed the drilling fluid parameters into the trained computing model 120 for predicting the gel strength of the drilling fluid at the second predetermined time interval in the real-time. The prediction module 204 may be configured to predict the gel strength of the drilling fluid at the second predetermined time interval by utilizing the trained computing model 120 based on the measurements of the drilling fluid parameters. The prediction module 204 may be configured to transmit the predicted gel strength measurements of the drilling fluid at the second predetermined time interval to the output module 206.

The output module 206 may be communicatively coupled to the prediction module 204. The output module 206 may be configured to receive the predicted gel strength measurements of the drilling fluid from the prediction module 204. The output module 206 may be configured to display the predicted gel strength measurements of the drilling fluid on the user interface 112 (as shown in the FIG. 1A) of the user device 102. The output module 206 may be configured to enable the operator to manually adjust the density of the drilling fluid to control suspension and removal of well cuttings based on the predicted gel strength measurements. In another embodiment, the output module 206 may be configured to automatically adjust the density of the drilling fluid based on the predicted gel strength measurements.

In an embodiment, the density of the drilling fluid may be adjusted by adding additives to the drilling fluid. The additives improve hole-cleaning properties of the drilling fluid, as well as plastic viscosity and the gel strength of the drilling fluid. In an embodiment, the additives may be, but not limited to, weighing agents, base fluid, thinners, and so forth. The weighing agents may be, but not limited to, Barite, siderite, calcium carbonate, hematite, ilmenite, galena, and so forth. As used herein, the term “Barite” is one of main weighting materials and is used to increase the density of the drilling mud to equalize pressure between the wellbore and formation when drilling through pressurized zones. Embodiments of the present invention include or otherwise cover any type of the weighing agents including known, related art, and/or later developed technologies.

Further, as used herein, the term “base fluid” may be added to the drilling mud to decrease solid concentration. This process is called dilution which is important to perform when new hole is drilled, and mud properties needs to be maintained as mud volume increases. Dilution volume is added to the drilling mud to control solids and to maintain volume. Maintaining a stable hole environment sustains hole integrity, controls reactive shale and maintains a consistent mud system when encountering contaminants.

In a preferred embodiment, the additives may be dispersants such as, but not limited to, Lignite, desco, lignosulfonate, and so forth that may be added to the drilling fluid to adjust rheology of the drilling fluid. As used herein, the term “dispersants” reduce the gel strength and help in controlling the rheology of the drilling mud. The dispersants thin the drilling mud by separating particles so they can be carried out of the hole by the drilling fluid. Embodiments of the present invention are intended to include or otherwise cover any type of the dispersants including known, related art, and/or later developed technologies. Embodiments of the present invention are intended to include or otherwise cover any type of the additives including known, related art, and/or later developed technologies.

FIG. 3 illustrates an exemplary schematic representation of the artificial neural network (ANN) model 300 for predicting the gel strength of the drilling fluid, according to certain embodiments. The artificial neural network (ANN) model 300 may be employed in the petroleum industry for various machine learning applications, particularly in predicting complex behaviours such as, the gel strength of the drilling fluid. As used herein, the term “artificial neural network (ANN) model 300” mimics biological neural networks, using interconnected nodes (neurons) that can process information and learn from the data.

According to an embodiment, the artificial neural network (ANN) model 300 may include the input layer 302, one or more hidden layers 304a-304m (hereinafter collectively referred to as the hidden layers 304 and individually referred to as the hidden layer 304), and the output layer 306. The input layer 302 may be designed to receive input data representing the measurements of the drilling fluid parameters. In an embodiment, each of the drilling fluid parameters may correspond to neurons 308a-308b in the input layer 302, enabling direct mapping of the measurements of the corresponding drilling fluid parameters to a structure of the artificial neural network (ANN) model 300. In an embodiment, the input layer 302 may be designed to provide the input data to subsequent hidden layers 304, where the input data is processed by a neural network. The input layer 302 ensures that the input data is standardized as required, enhancing compatibility with the weight of the neural network and the transfer functions.

Further, the hidden layer 304 may include interconnected neurons 310a-310p (hereinafter collectively referred to as the neurons 310 and individually referred to as the neuron 310) that processes the input data through weighted connections and the transfer functions. Each of the neurons 310 in the hidden layer 304 may apply a weighted sum of the input data and passes a result of the weighted sum to the transfer function, enhancing an ability to capture a complex function. In an exemplary embodiment, the artificial neural network (ANN) model 300 may employ the single hidden layer 304 with 18 neurons 310, each applying the log-sigmoid (log sig) transfer function between the input layer 302 and the hidden layer 304. This log-sigmoid (log sig) transfer function scales and normalizes the input data as the input data is processed by each neuron 310 of the hidden layer 304, providing a non-linear mapping that captures intricate patterns within the input data.

Further, the hidden layer 304 connects to the output layer 306, where a single neuron 312 of the output layer 306 aggregates the processed input data and applies the pure-linear (purelin) transfer function between the hidden layer 304 and the output layer 306 to produce the output. This pure-linear (purelin) transfer function between the hidden layer 304 and the output layer 306 ensures that a final prediction directly aligns with the actual gel strength measurements, allowing accurate predictions that represents the predicted gel strength measurements at the second predetermined time interval.

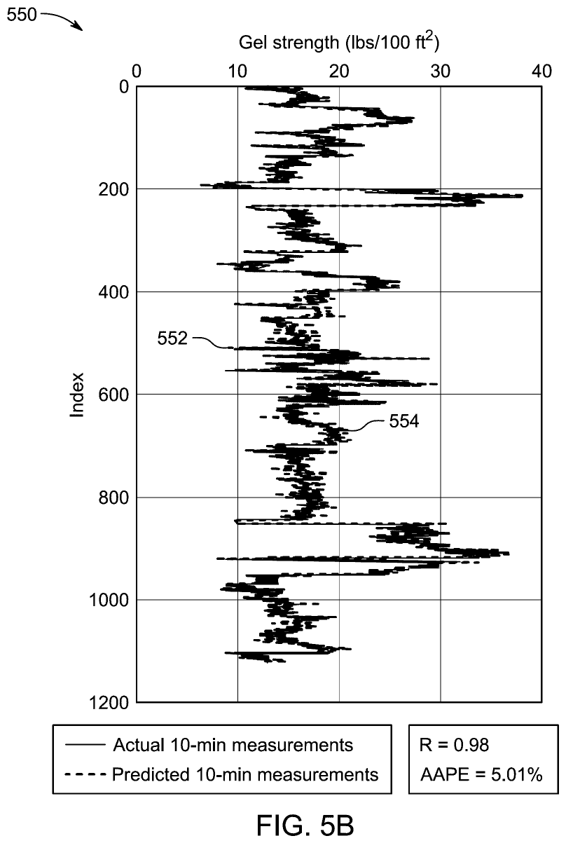

FIG. 4 illustrates a graph 400 showing the correlation coefficients (R) between model input parameters and model output, according to certain embodiments. In an embodiment, analysis of the correlation coefficients (R) provides insights into relationships between the model input parameters and the model output. The model input parameters may be the drilling fluid parameters such as, the mud weight (MW) and drilling fluid viscosity. The model output may be the measurements of the gel strength of the drilling fluid. Herein, the mud weight (MW) displays a positive correlation with the gel strength measurements with correlation coefficients (R)-values of 0.39 for 10-second reading and 0.36 for 10-minute reading, which indicates that an increase in the mud weight (MW) generally corresponds to an increase in both a short-term strength (i.e. 10-second gel strength) and a long-term gel strength (i.e. 10-minute gel strength). In contrast, the drilling fluid viscosity shows a weak inverse relationship with the gel strength measurements, where the correlation coefficients (R)-values of −0.07 for the 10-second reading and −0.17 for the 10-minute reading, implying that a higher viscosity tends to correlate with a lower gel strength. Hence, inverse relationships indicate that as a drilling fluid viscosity time increases, the ability of the drilling fluid to develop the gel strength diminishes slightly.

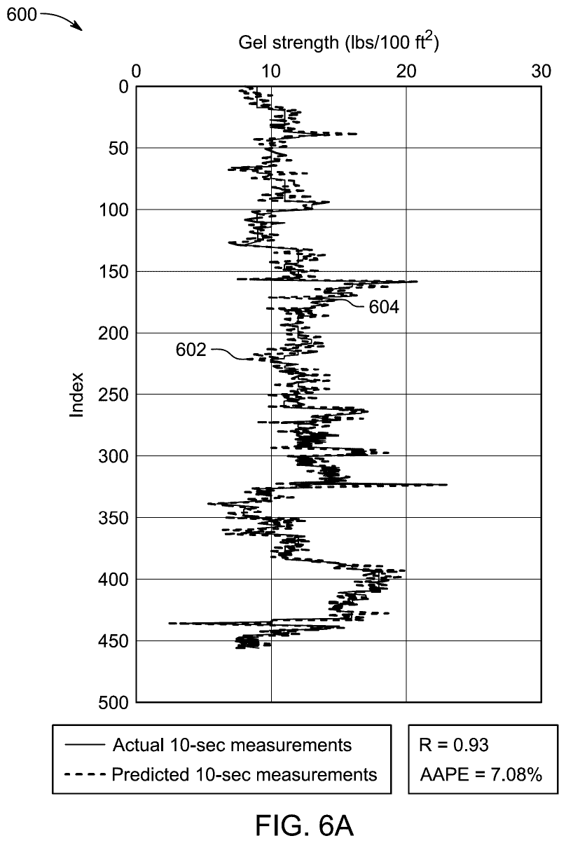

FIGS. 5A-5B illustrates a graph 500 representing a comparison between the predicted gel strength measurements and the actual gel strength measurements for the training data, according to certain embodiments. Here, the computing model 120 (as shown in the FIG. 1A) may be trained to predict the gel strength measurements at the second predetermined time interval (i.e. the 10-second interval and the 10-minute interval) using the mud weight (MW) and the drilling fluid viscosity measurements as the inputs. The training data may include 1,022 data points, while a separate testing data of 455 data points may be used to evaluate predictive capabilities of the computing model 120. This segregation ensured that the testing data remains independent of a training process, allowing for an unbiased assessment of the model accuracy.

During training, the hyperparameters of the computing model 120 may be optimized to minimize a prediction error by adjusting for both the mud weight (MW) and the drilling fluid viscosity measurements. Upon training, the computing model 120 may be tested on the testing data to assess the performance on the unseen data. The predicted gel strength measurements may be compared against the actual gel strength measurements, using the performance metrics such as the correlation coefficient (R) and the average absolute percentage error (AAPE). These metrics provided insight into the accuracy of the computing model 120 and ability to generalize to new data.

Referring to FIGS. 5A and 5B, results indicate a strong alignment between the predicted gel strength measurements and the actual measured gel strength measurements, as reflected in high correlation coefficients (R) of 0.93 for the 10-second gel strength prediction and 0.98 for the 10-minute gel strength prediction. These correlation coefficients (R) values imply that the computing model 120 effectively captures the relationship between the mud weight (MW), the drilling fluid viscosity, and the gel strength, achieving high accuracy in its predictions.

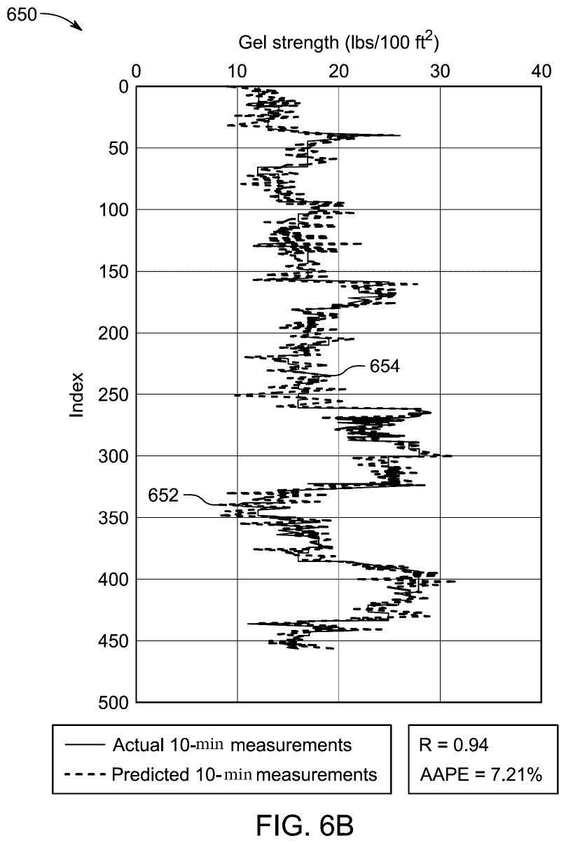

Additionally, low average absolute percentage error (AAPE) percentages such as, 6.76% for the 10-second gel strength and 5.01% for the 10-minute gel strength indicate a precision of the computing model 120. Lower average absolute percentage error (AAPE) percentages indicate that the predictions of the computing model 120 are close to the actual gel strength measurements, with a minimal deviation. The alignment and accuracy observed in FIGS. 5A and 5B confirm a capability of the computing model 120 to reliably predict the gel strength across different datasets and time intervals, supporting its suitability for deployment in real-world applications. FIGS. 6A-6B illustrates a graph 600 representing a comparison between the predicted gel strength measurements and the actual gel strength measurements for the testing data, according to certain embodiments. FIG. 6 illustrates testing results for the gel strength predictions using the artificial neural network (ANN) model 300, evaluated with the unseen data consisting of 650 data points. Here, the computing model 120 demonstrated strong predictive capabilities, with the correlation coefficients (R)-value exceeding 0.93, indicating a high degree of correlation between the predicted gel strength measurements and the actual gel strength measurements. The average absolute percentage error (AAPE) for predictions ranged from 7.08% to 7.21%, confirming the ability of the computing model 120 to produce the accurate predictions with the minimal error.

Further, predicted gel strength profiles, as shown in the FIGS. 6A and 5B, validate the performance of the computing model 120, providing a visual comparison between the predicted gel strength measurements and the actual gel strength measurements. These results underline a reliability of the artificial neural network (ANN) model 300 in accurately forecasting the gel strength for real-world drilling applications, with statistical metrics such as the correlation coefficient (R) and the average absolute percentage error (AAPE) supporting an effectiveness of the computing model 120 for practical deployment.

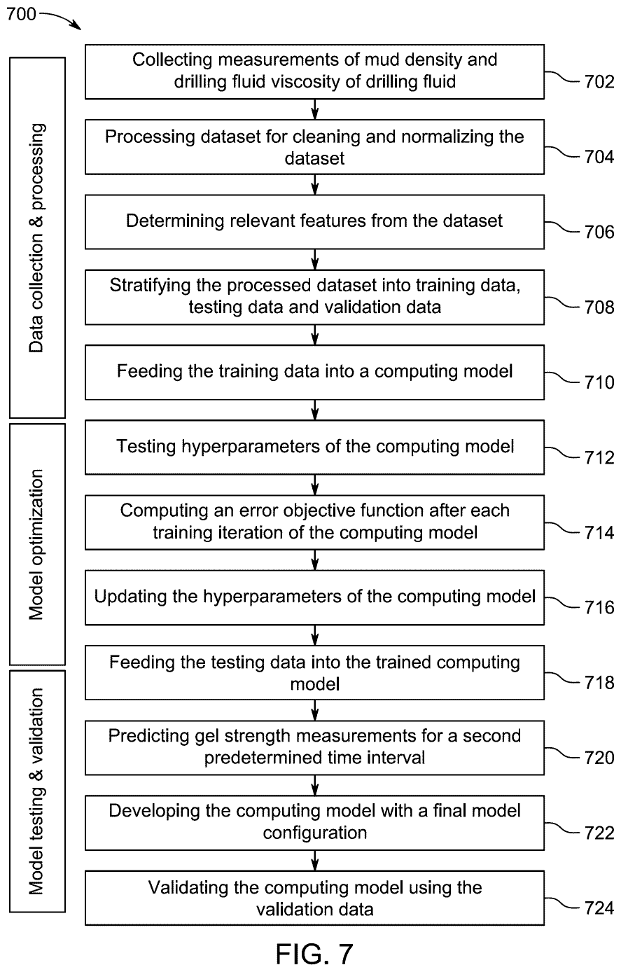

FIG. 7 illustrates a flowchart of a method 700 for developing the computing model 120, according to certain embodiments. The method 700 includes a series of steps. These steps are only illustrative, and other alternatives may be considered where one or more steps are added, one or more steps are removed, or one or more steps are provided in a different sequence without departing from the scope of the present disclosure.

At step 702, the method 700 includes collecting the dataset containing the historical measured values of the drilling fluid parameters and the actual gel strength measurements from the database 118. In an exemplary embodiment, the historical measured values of the drilling fluid parameters may be used as the model inputs and the actual gel strength measurements may be considered as the target output. The drilling fluid parameters and the actual gel strength measurements may be collected from the database 118 by establishing the connection with the database 118 via the interface.

At step 704, the method 700 includes processing the dataset for removing the errors, the duplicates, the inconsistencies, and so forth. This step involves handling the missing values, the outliers, and the noisy data by using the data cleaning techniques. This step also involves normalizing the dataset by using normalization techniques. The dataset may be normalized for maintaining the comparability, if measurements of the drilling fluid parameters differ in the units or the scales.

At step 706, the method 700 includes retrieving the relevant features such as, the mud weight (MW), and the drilling fluid viscosity from the dataset

At step 708, the method 700 includes stratifying the processed dataset into the training data, the testing data and the validation data by using the stratification technique. Herein, the stratification technique may be the stratified dataset splitting.

At step 710, the method 700 includes feeding the training data into the computing model 120 for generating the baseline computing model 120 based on the default hyperparameters. The baseline computing model 120 may help in establishing the performance benchmark for comparison for the subsequent optimization steps.

At step 712, the method 700 includes testing the hyperparameters of the computing model 120 for determining the optimal hyperparameter combination for the computing model 120. This step involves determining the combinations of the hyperparameters by using the optimization technique and performing training on the computing model 120 with each of the combinations of the hyperparameters. Herein, the hyperparameters may be, but not limited to, the learning rate, the number of hidden layers 304, the number of neurons 310 per hidden layer 304, the transfer functions, the training algorithms, and so forth. Herein, the optimization technique may be the grid search function. In an aspect, the performance of the computing model 120 is assessed on each of the combinations of the hyperparameters to identify the optimal combination of the hyperparameters.

At step 714, the method 700 includes computing the error objective function after each training iteration for performing the weight adjustments within the computing model 120. This step involves iteratively training the computing model 120 on the training data with the optimal combination of the hyperparameters for predicting the gel strength of the drilling fluid. For each prediction, the error objective function is calculated by taking the difference between the actual gel strength measurements and the predicted gel strength measurements, squaring the result of the difference, and averaging across all the samples of the training data.

At step 716, the method 700 involves updating the hyperparameters of the computing model 120 based on the computed error objective function to improve the prediction accuracy.

At step 718, the method 700 involves feeding the testing data into the trained computing model 120 to predict the target output for the second predetermined time interval. In an aspect, the second predetermined time interval may be the 10-second interval. In another aspect, the second predetermined time interval may be the 10-minute interval.

At step 720, the method 700 includes predicting the gel strength measurements for the second predetermined time interval using the testing data to evaluate the performance of the computing model 120 on the unseen data. The performance metrics such as, but not limited to, the correlation coefficient (R), the average absolute percentage error (AAPE), and so forth may be recorded for evaluating the prediction accuracy of the computing model 120.

At step 722, the method 700 includes developing the computing model 120 with the final model configuration. This step involves analyzing the testing performance of the computing model 120 by reviewing the values of the performance metrics. If the performance metrics do not meet the predefined accuracy thresholds, then the iterative refinements may be performed until the computing model 120 achieves the desired performance. Once the iterative refinements are completed and the desired performance is achieved, then the computing model 120 may be developed with the final model configuration. Herein, the final model configuration may include the single hidden layer 304 containing the 18 neurons 310, the levenberg-marquardt backpropagation (trainlm) algorithm with the learning rate of 0.10, utilizing the log-sigmoid (log sig) transfer function between the input layer 302 and the hidden layers 304, and the pure-linear (purelin) transfer function between the hidden layer 304 and the output layer 306.

At step 724, the method 700 includes validating the computing model 120 using the validation data for confirming the predictive performance on the new data. This step involves calculating the performance metrics such as, but not limited to, the correlation coefficient (R) and the average absolute percentage error (AAPE) to ensure that the predictive accuracy of the computing model 120 meets the predefined deployment criteria.

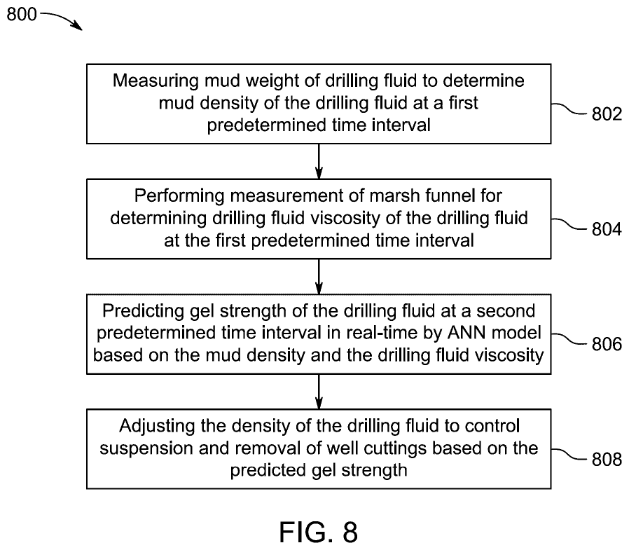

FIG. 8 illustrates a flowchart of a method 800 for predicting the gel strength measurements of the drilling fluid, according to certain embodiments. The method 800 includes a series of steps. These steps are only illustrative, and other alternatives may be considered where one or more steps are added, one or more steps are removed, or one or more steps are provided in a different sequence without departing from the scope of the present disclosure.

At step 802, the method 800 includes measuring the mud weight (MW) of the drilling fluid to determine the mud density of the drilling fluid at the first predetermined time interval. The mud weight (MW) of the drilling fluid may be measured by using the mud balance 124. In an aspect, the drilling fluid may be the synthetic oil-based mud. In an aspect, the first predetermined time interval may be every hour. In another aspect, the first predetermined time interval may be after a few hours.

At step 804, the method 800 includes performing the measurement of the marsh funnel (MF) 134 for determining the drilling fluid viscosity of the drilling fluid at the first predetermined time interval.

At step 806, the method 800 includes predicting the gel strength of the drilling fluid at the second predetermined time interval in the real-time by the artificial neural network (ANN) model 300 based on the mud density and the drilling fluid viscosity. In an aspect, the second predetermined time interval may be the 10-second interval. In another aspect, the second predetermined time interval may be the 10-minute time interval. The prediction of the gel strength of the drilling fluid may be performed by feeding the measurements of the mud density and the drilling fluid viscosity as the input data into the input layer 302 of the artificial neural network (ANN) model 300. The input layer 302 may be designed to provide the input data to the subsequent hidden layers 304, where the input data is processed and transformed by the neural network. The interconnected neurons 310 of the hidden layer 304 processes and transforms the input data through the weighted connections and the transfer functions. In an exemplary embodiment, the artificial neural network (ANN) model 300 may employ the single hidden layer 304 with the 18 neurons 310, each applying the log-sigmoid (log sig) transfer function between the input layer 302 and the hidden layer 304 to scale and normalize the input data. After the input data is processed by the hidden layer 304, transformed activations are passed to the output layer 306 of the artificial neural network (ANN) model 300. The output layer 306 applies the pure-linear (purelin) transfer function between the hidden layer 304 and the output layer 306 to combine the activations from the hidden layer 304 and generates the predicted gel strength measurements.

At step 808, the method 800 includes adjusting the density of the drilling fluid to control the suspension and the removal of well cuttings based on the predicted gel strength. The density of the drilling fluid may be adjusted by adding the additives to the drilling fluid. In an aspect, the additives may be the dispersants such as, but not limited to, the Lignite, the desco, the lignosulfonate, and so forth that may be added to the drilling fluid to adjust the rheology of the drilling fluid.

In an aspect, the method 800 includes performing the drilling operation in the drilling fluid by the drilling rig 122. In an aspect, the drilling operation is performed in the vertical well. In another aspect, the drilling operation is performed in the horizontal well. In yet another aspect, the drilling operation is performed in the inclined well. In another aspect, the drilling operation is performed in the multilateral well.

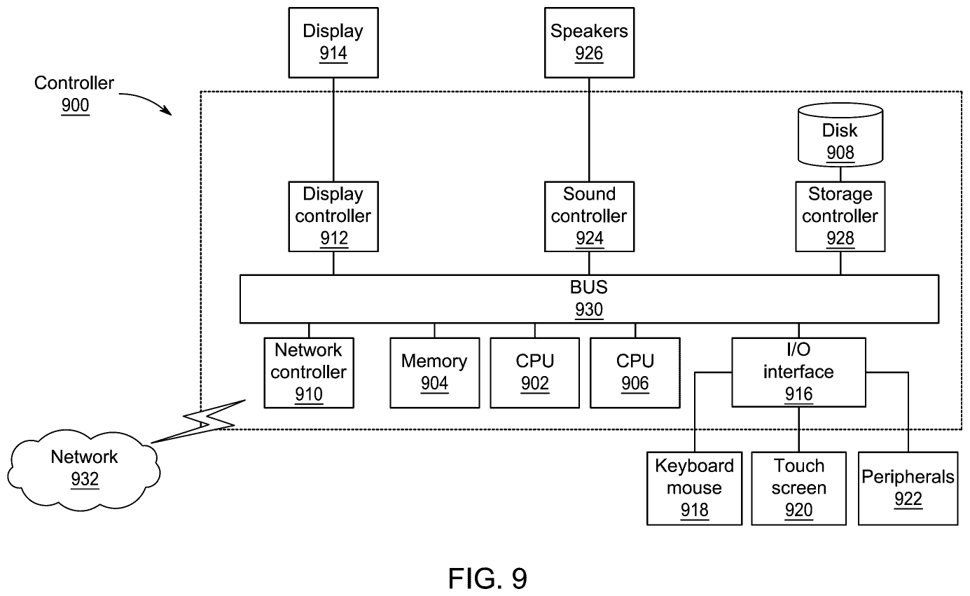

FIG. 9 is an illustration of a non-limiting example of details of a computing hardware used in a computing system, according to certain embodiments. In the FIG. 9, a controller 900 is described as representative of the system 100 in which the controller 900 is a computing device which includes a central processing unit (CPU) 902 which performs processes described above/below. The process data and instructions may be stored in a memory 904. These processes and instructions may also be stored on a storage medium disk 908 such as a hard drive (HDD) or a portable storage medium or may be stored remotely.

Further, the present disclosure is not limited by the form of the computer-readable media on which the instructions of the inventive process are stored. For example, the instructions may be stored on compact discs (CDs), digital versatile disc (DVDs), in FLASH memory, read access memory (RAM), read only memory (ROM), programmable read only memory (PROM), erasable programmable read only memory (EPROM), electrically erasable programmable read only memory (EEPROM), hard disk or any other information processing device with which the computing device communicates, such as a server or computer.

Further, the present disclosure may be provided as a utility application, background daemon, or component of an operating system, or combination thereof, executing in conjunction with CPU 902, 906 and an operating system such as Microsoft Windows 10, Microsoft Windows 11, UNiplexed Information Computing System (UNIX), Solaris, Lovable Intellect Not Using XP (LINUX), Apple Macintosh (MAC)-Operating System (OS) and other systems known to those skilled in the art.

The hardware elements in order to achieve the computing device may be realized by various circuitry elements, known to those skilled in the art. For example, CPU 902 or CPU 906 may be a Xenon or Core processor from Intel of America or an Opteron processor from advanced micro devices (AMD) of America, or may be other processor types that would be recognized by one of ordinary skill in the art. Alternatively, the CPU 902, 906 may be implemented on a field programmable Gate array (FPGA), application-specific integrated circuit (ASIC), programmable logic device (PLD) or using discrete logic circuits, as one of ordinary skill in the art would recognize. Further, CPU 902, 906 may be implemented as multiple processors cooperatively working in parallel to perform the instructions of the inventive processes described above.

The computing device in the FIG. 9 also includes a network controller 910, such as an Intel Ethernet PRO network interface card from Intel Corporation of America, for interfacing with network 932. As can be appreciated, the network 910 can be a public network, such as the Internet, or a private network such as a local area network (LAN) or a wide area network (WAN) network, or any combination thereof and can also include public switched telephone network, (PSTN) or an integrated services digital network (ISDN) sub-networks. The network 932 can also be wired, such as an Ethernet network, or can be wireless such as a cellular network including EDGE, 3G and 4G wireless cellular systems. The wireless network can also be Wireless Fidelity (WiFi), Bluetooth, or any other wireless form of communication that is known.

The computing device further includes a display controller 912, such as a NVIDIA GeForce GTX or Quadro graphics adaptor from NVIDIA Corporation of America for interfacing with display 914, such as a Hewlett Packard HPL2445w LCD monitor. A general purpose I/O interface 916 interfaces with a keyboard and/or mouse 918 as well as a touch screen panel 920 on or separate from display 914. General purpose I/O interface also connects to a variety of peripherals 922 including printers and scanners, such as an OfficeJet or DeskJet from Hewlett Packard.

A sound controller 924 is also provided in the computing device such as Sound Blaster X-Fi Titanium from Creative, to interface with speakers/microphone 926 thereby providing sounds and/or music.

The general purpose storage controller 928 connects the storage medium disk 908 with communication bus 930, which may be an instruction set architecture (ISA), extended industry standard architecture (EISA), video electronics standards association (VESA), peripheral component interconnect (PCI), or similar, for interconnecting all of the components of the computing device. A description of the general features and functionality of the display 914, keyboard and/or mouse 918, as well as the display controller 912, storage controller 928, network controller 910, sound controller 924, and general purpose I/O interface 916 is omitted herein for brevity as these features are known.

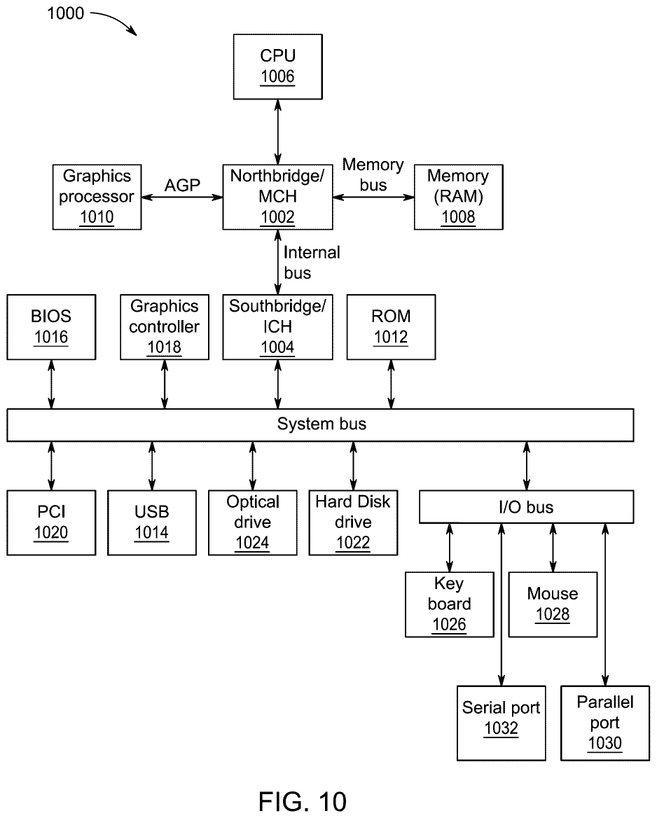

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 10.

FIG. 10 is an exemplary schematic diagram of a data processing system 1000 used within the computing system, according to certain embodiments, for performing the functions of the exemplary embodiments. The data processing system 1000 is an example of a computer in which code or instructions implementing the processes of the illustrative embodiments may be located.

In the FIG. 10, the data processing system 1000 employs a hub architecture including a north bridge and memory controller hub (NB/MCH) 1002 and a south bridge and input/output (I/O) controller hub (SB/ICH) 1004. The central processing unit (CPU) 1006 is connected to the NB/MCH 1002. The NB/MCH 1002 also connects to the memory 1008 via a memory bus, and connects to the graphics processor 1010 via an accelerated graphics port (AGP). The NB/MCH 1002 also connects to the SB/ICH 1004 via an internal bus (e.g., a unified media interface or a direct media interface). The CPU 1006 may contain one or more processors and even may be implemented using one or more heterogeneous processor systems.

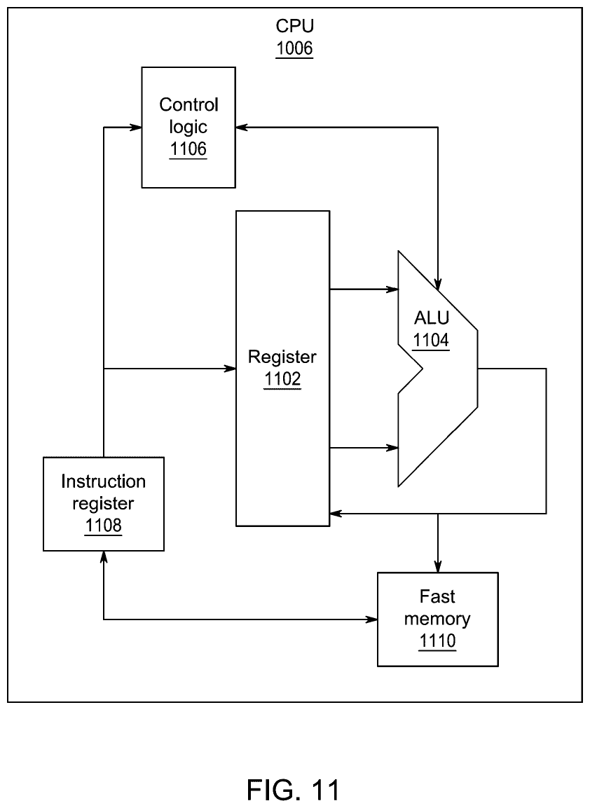

For example, FIG. 11 shows one implementation of the CPU 1006. In one implementation, the instruction register 1108 retrieves instructions from the fast memory 1110. At least part of these instructions are fetched from the instruction register 1108 by the control logic 1106 and interpreted according to the instruction set architecture of the CPU 1006. Part of the instructions can also be directed to the register 1102. In one implementation the instructions are decoded according to a hardwired method, and in another implementation the instructions are decoded according a microprogram that translates instructions into sets of CPU configuration signals that are applied sequentially over multiple clock pulses. After fetching and decoding the instructions, the instructions are executed using the arithmetic logic unit (ALU) 1104 that loads values from the register 1102 and performs logical and mathematical operations on the loaded values according to the instructions. The results from these operations can be feedback into the register 1102 and/or stored in the fast memory 1110. According to certain implementations, the instruction set architecture of the CPU 1006 can use a reduced instruction set architecture, a complex instruction set architecture, a vector processor architecture, a very large instruction word architecture. Furthermore, the CPU 1006 can be based on a Von Neuman model or a Harvard model. The CPU 1006 can be a digital signal processor, the FPGA, the ASIC, the PLA, a PLD, or a CPLD. Further, the CPU 1006 can be an x86 processor by Intel or by AMD; an ARM processor, a Power architecture processor by, e.g., IBM; a SPARC architecture processor by Sun Microsystems or by Oracle; or other known CPU architecture.

Referring again to the FIG. 10, the data processing system 1000 can include that the SB/ICH 1004 is coupled through a system bus to an I/O Bus, a read only memory (ROM) 1012, universal serial bus (USB) port 1014, a flash binary input/output system (BIOS) 1016, and a graphics controller 1018. PCI/PCIe devices can also be coupled to SB/ICH 1004 through a PCI bus 1020.

The PCI devices may include, for example, Ethernet adapters, add-in cards, and PC cards for notebook computers. The Hard disk drive 1022 and CD-ROM 1024 can use, for example, an integrated drive electronics (IDE) or serial advanced technology attachment (SATA) interface. In one implementation the I/O bus can include a super I/O (SIO) device.

Further, the hard disk drive (HDD) 1022 and optical drive 1024 can also be coupled to the SB/ICH 1004 through a system bus. In one implementation, a keyboard 1026, a mouse 1028, a parallel port 1030, and a serial port 1032 can be connected to the system bus through the I/O bus. Other peripherals and devices that can be connected to the SB/ICH 1004 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

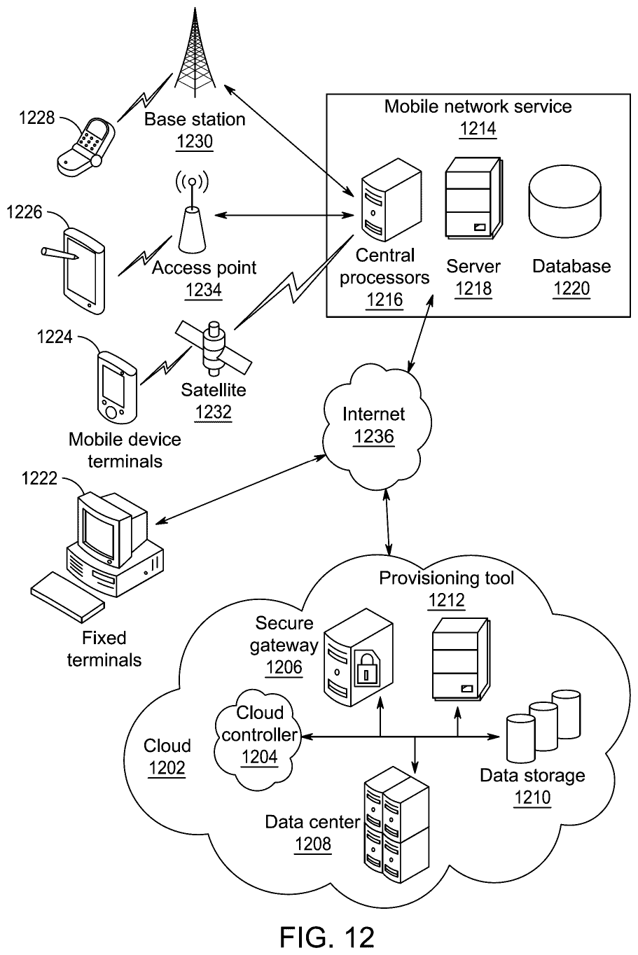

The functions and features described herein may also be executed by various distributed components of a system. For example, one or more processors may execute these system functions, wherein the processors are distributed across multiple components communicating in a network. The distributed components may include one or more client and server machines, such as cloud 1202 including a cloud controller 1204, a secure gateway 1206, a data center 1208, data storage 1210 and a provisioning tool 1212, and mobile network services 1214 including central processors 1216, a server 1218 and a database 1220, which may share processing, as shown by FIG. 12, in addition to various human interface and communication devices (e.g., display monitors 1222, smart phones 1224, tablets 1226, personal digital assistants (PDAs) 1228). The network may be a private network, such as a base station 1230, satellite 1232 or access point 1234, or be a public network, may such as the Internet 1236. Input to the system 100 may be received via direct user input and received remotely either in real-time or as a batch process. Additionally, some implementations may be performed on modules or hardware that are not identical to those described. Accordingly, other implementations are within the scope of the present disclosure.

The above-described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

1. A computer-implemented method for monitoring and treatment of a drilling fluid during a drilling operation, comprising:
measuring a mud weight (MW) of the drilling fluid to determine a mud density of the drilling fluid at a first predetermined time interval;
performing a Marsh funnel (MF) measurement to determine a drilling fluid viscosity of the drilling fluid at the first predetermined time interval;
predicting a gel strength of the drilling fluid for a second predetermined time interval in real-time by an artificial neural network based on the mud density and the drilling fluid viscosity; and
adjusting density of the drilling fluid to control suspension and removal of well cuttings by adding an additive based on the predicted gel strength.

2. The method of claim 1, further comprising predicting the gel strength for the second predetermined time interval of 10 seconds by the artificial neural network.

3. The method of claim 1, further comprising predicting gel strength for the second predetermined time interval of 10 minutes by the artificial neural network.

4. The method of claim 1, wherein the drilling fluid is synthetic oil, and wherein the adding includes adding a dispersant to adjust rheology of the drilling fluid.

5. The method of claim 1, wherein the artificial neural network is configured with a single hidden layer of 18 neurons, the method includes
applying a log-sigmoidal transfer function between an input layer and the hidden layer, and
applying a pure-linear transfer function between the hidden and output layers.

6. The method of claim 1, further comprising drilling in a vertical well.

7. The method of claim 1, further comprising drilling in a horizontal well.

8. The method of claim 1, further comprising drilling in an inclined well.

9. The method of claim 1, further comprising drilling in a multilateral well.

10. The method of claim 4, further comprising adding one of Lignite, desco, and lignosulfonate as the dispersant to adjust rheology of the drilling fluid.

11. A computer-implemented system for monitoring and treatment of a drilling fluid during a drilling operation, comprising:
a drilling rig configured to perform the drilling operation with the drilling fluid;
a mud balance to measure mud weight (MW) of the drilling fluid at a first predetermined time interval;
a Marsh funnel (MF) to determine a drilling fluid viscosity of the drilling fluid at the first predetermined time interval;
processing circuitry configured with an artificial neural network to predict a gel strength of the drilling fluid for a second predetermined time interval in real time based on the mud weight (MW) and the drilling fluid viscosity, and
adjust density of the drilling fluid to control suspension and removal of well cuttings with a dispenser for adding an additive in accordance with the predicted gel strength.

12. The system of claim 11, wherein the artificial neural network predicts the gel strength for the second predetermined time interval of 10 seconds.

13. The system of claim 11, wherein the artificial neural network predicts the gel strength for the second predetermined time interval of 10 minutes.

14. The system of claim 11, wherein the drilling fluid is synthetic oil, and wherein the adding includes adding a dispersant to adjust rheology of the drilling fluid.

15. The system of claim 11, wherein the artificial neural network is configured with a single hidden layer of 18 neurons,
to apply a log-sigmoidal transfer function between an input layer and the hidden layer, and
to apply a pure-linear transfer function between the hidden and output layers.

16. The system of claim 11, wherein the drilling rig is configured to drill in a vertical well.

17. The system of claim 11, wherein the drilling rig is configured to drill in a horizontal well.

18. The system of claim 11, wherein the drilling rig is configured to drill in an inclined well.

19. The system of claim 11, wherein the drilling rig is configured to drill in a multilateral well.

20. The system of claim 14, wherein the dispenser adds one of Lignite, desco, and lignosulfonate as the dispersant to adjust rheology of the drilling fluid.
